(19) United States
(12) Patent Application Publication
(10) Pub. No.: US 2025/0285733 A1
(43) Pub. Date: Sep. 11, 2025

(54) REMOTE ELECTRICAL STIMULATION SYSTEM

(71) Applicant: GOLDEN SMART HOME TECHNOLOGY CORP., Hsinchu City (TW)

(72) Inventors: Fang-Cheng Liu, Hsinchu City (TW); Chuan-Fang Lin, Zhubei City (TW); Tung-Shu YANG, Toufen City (TW)

(21) Appl. No.: 19/075,379

(22) Filed: Mar. 10, 2025

Related U.S. Application Data
(60) Provisional application No. 63/563,720, filed on Mar. 11, 2024.

(30) Foreign Application Priority Data
Jul. 22, 2024 (TW) 113127313
Jul. 22, 2024 (TW) 113207850

(51) Int. Cl. G16H 20/30; A61N 1/04; A61N 1/36

(52) U.S. Cl. CPC G16H 20/30; A61N 1/0452; A61N 1/0492; A61N 1/36034

(57) ABSTRACT

Provided is a remote electrical stimulation system, including a cloud platform, a patient operating terminal, and an electrical stimulation device. The cloud platform includes a prescription database, a prescription editing module, and a prescription specifying module. The prescription database is configured to store a plurality of electrical stimulation prescriptions. The prescription editing module is configured to receive editing instructions to edit or create an electrical stimulation prescription, and the prescription specifying module is configured to receive specifying instructions to designate one of the plurality of electrical stimulation prescriptions as an exclusive electrical stimulation prescription that can only be downloaded by a target patient. The electrical stimulation device is configured to receive the exclusive electrical stimulation prescription from the patient operating terminal and utilize it to perform an electrical stimulation treatment on the target patient.

### CROSS REFERENCE TO RELATED APPLICATIONS

This patent application claims priority to Patents Application Nos. 113127313, and 11320785 both filed in Taiwan on Jul. 22, 2024, and this application claims priority of U.S. Provisional Application No. 63/563,720 filed on Mar. 11, 2024 under 35 U.S.C. § 119(e), the entire contents of all which are incorporated herein by reference.

### BACKGROUND

### Technical Field

The present disclosure relates to an electrical stimulation system, and more particularly to a remote electrical stimulation system that enables remote operation and execution.

### Description of Related Art

Electrical stimulation primarily utilizes physical electrical signals, which are introduced into a patient's body through the patient's skin to stimulate an affected area, thereby producing a therapeutic effect on the muscles and/or nerves of the affected area. Traditionally, a patient who requires electrical stimulation has to frequently visit a medical institution to obtain an electrical stimulation prescription from a physician and regularly use dedicated electrical stimulation equipment within the medical institution to undergo the corresponding electrical stimulation treatment. However, most patients who require electrical stimulation therapy often experience mobility difficulties due to limb pain, weakness, or impairment. If a patient is not hospitalized, it may be needed for the patient to frequently commute between home and the medical institution, which imposes significant transportation and time costs on both the patient and the patient's family. This added burden on daily life may further reduce the patient's willingness to actively pursue treatment, thereby diminishing the effectiveness of the therapy.

Accordingly, designing a remote electrical stimulation system that can effectively address the aforementioned issues is an urgent problem that needs to be solved.

### SUMMARY

The present disclosure provides a remote electrical stimulation system that utilizes a cloud platform as a medium, enabling remote execution of electrical stimulation prescription therapy between physicians and patients, thereby reducing the number of visits and the time patients would otherwise have to spend receiving treatment at medical institutions.

The present disclosure, based on the aforementioned remote electrical stimulation system, allows physicians to utilize a user interface provided by the cloud platform to independently select, edit, or adjust electrical stimulation prescriptions according to medical conditions or status of different patients and to upload and store electrical stimulation prescriptions to the cloud platform.

The present disclosure, based on the aforementioned remote electrical stimulation system, may also allow an electronic device, such as a mobile phone or a computer, to download the patient's exclusive electrical stimulation prescription from the cloud platform and communicate with the electrical stimulation device, thereby enabling the patient to use the electrical stimulation device to execute the exclusive electrical stimulation prescription and achieve the therapeutic effects of electrical stimulation.

In at least one embodiment, the remote electrical stimulation system of the present disclosure includes a cloud platform, a patient operating terminal, and an electrical stimulation device. In some embodiments of the present disclosure, the cloud platform includes a prescription database, a prescription editing module, and a prescription specifying module, wherein the prescription database is configured to store a plurality of electrical stimulation prescriptions, the prescription editing module is configured to receive editing instructions to edit or create an electrical stimulation prescription, and the prescription specifying module is configured to receive specifying instructions to designate one of the plurality of electrical stimulation prescriptions as an exclusive electrical stimulation prescription that can only be downloaded by a target patient who has download authorization for the exclusive electrical stimulation prescription. In some embodiments of the present disclosure, subject to current medical regulations, the patient operating terminal requires authorized personnel to assist a target patient in downloading the exclusive electrical stimulation prescription from the cloud platform. Once telemedicine is approved for relevant use in the future, the target patient will be able to download the exclusive electrical stimulation prescription from the cloud platform independently. In some embodiments of the present disclosure, the electrical stimulation device is configured to receive the exclusive electrical stimulation prescription from the patient operating terminal and utilize the exclusive electrical stimulation prescription to perform an electrical stimulation treatment on the target patient. In some embodiments, subject to current medical regulations, the authorized personnel of the present disclosure include physicians and other medical-related personnel. In other embodiments, once telemedicine is approved for relevant use in the future, the authorized personnel of the present disclosure may further include the target patients themselves.

In at least one embodiment of the present disclosure, the electrical stimulation device includes a first patch set corresponding to a first channel and a second patch set corresponding to a second channel, and the exclusive electrical stimulation prescription includes a first carrier signal and a second carrier signal, wherein the electrical stimulation device is configured to transmit the first carrier signal to the first patch set via the first channel and transmit the second carrier signal to the second patch set via the second channel.

In at least one embodiment of the present disclosure, the first carrier signal and the second carrier signal correspond to different carrier waveform combinations and/or timing sequences.

In at least one embodiment of the present disclosure, the first carrier signal and the second carrier signal correspond to different carrier waveforms at the same time.

In at least one embodiment of the present disclosure, the cloud platform further comprises a login module configured to allow a user to log in and identify identity of the logged-in user to correspondingly grant at least one of an authorization for prescription editing, an authorization for prescription specifying, and an authorization for downloading the exclusive electrical stimulation prescription.

In at least one embodiment of the present disclosure, when the login module determines that a user has the authorization for prescription editing, the prescription editing module is configured to generate a prescription editing interface and receive the editing instructions via the prescription editing interface to edit or create the electrical stimulation prescription.

In at least one embodiment of the present disclosure, the prescription editing module includes a carrier waveform library, and the prescription editing interface includes a waveform selection area and an editing area, wherein the waveform selection area is configured to display carrier waveform templates stored in the carrier waveform library and to adjust or modify settings of any of the carrier waveform templates based on the editing instructions, and wherein the editing area is configured to select and edit either a single carrier waveform template or a combination of multiple carrier waveform templates from the waveform selection area based on the editing instructions or configured to draw a new carrier waveform based on the editing instructions.

In at least one embodiment of the present disclosure, the waveform selection area is configured to set at least one of type, frequency, and attenuation ratio of the carrier waveform for each displayed carrier waveform template.

In at least one embodiment of the present disclosure, the editing area includes a first carrier signal area and a second carrier signal area, wherein the first carrier signal area is configured to display, along a horizontal time axis, the first carrier signal of a single electrical stimulation prescription and allow editing of each carrier waveform in the first carrier signal for a corresponding position and duration on the time axis, and the second carrier signal area is configured to display, along the time axis, the second carrier signal of the single electrical stimulation prescription and allow editing of each carrier waveform in the second carrier signal for a corresponding position and duration on the time axis.

In at least one embodiment of the present disclosure, when the login module determines that the user has the authorization for prescription specifying, the prescription specifying module is configured to generate a prescription specifying interface and receive the specifying instructions via the prescription specifying interface to specify the exclusive electrical stimulation prescription that can only be downloaded by the target patient.

In at least one embodiment of the present disclosure, the cloud platform further comprises a learning module configured to compile historical records of each specified exclusive electrical stimulation prescription and the corresponding medical history data of the target patient for comparative learning and provide recommendation information for selecting the exclusive electrical stimulation prescription in the prescription specifying interface for different patients.

In at least one embodiment of the present disclosure, the electrical stimulation device further comprises a plurality of sensors configured to obtain feedback information from the target patient in using the exclusive electrical stimulation prescription to perform the electrical stimulation treatment and configured to transmit the feedback information back to the cloud platform via the patient operating terminal.

### BRIEF DESCRIPTION OF DRAWINGS

By reading the following descriptions of the embodiments and referring to the accompanying drawings, the present disclosure can be more fully understood.

FIG. 1 is a schematic diagram of the remote electrical stimulation system of the present disclosure.

FIG. 2 is a system block diagram of the remote electrical stimulation system of the present disclosure.

FIG. 3 is a schematic diagram of the prescription editing interface of the remote electrical stimulation system of the present disclosure.

FIG. 4 is a system block diagram of another embodiment of the remote electrical stimulation system of the present disclosure.

FIG. 5 is a system block diagram of yet another embodiment of the remote electrical stimulation system of the present disclosure.

### DETAILED DESCRIPTION

Since the various aspects and embodiments described herein are merely illustrative and not restrictive, those skilled in the art may understand that other aspects and embodiments are possible without departing from the scope of the present disclosure after reading this specification. The following detailed description and the claims will further highlight the features and advantages of these embodiments.

In the present disclosure, the use of “a” or “an” to describe components and elements herein is merely for convenience of explanation and to provide a general meaning regarding the scope of the present disclosure. Accordingly, unless explicitly stated otherwise, such descriptions should be understood to mean “one or at least one,” and the singular form also encompasses the plural form.

In the present disclosure, the terms “comprise,” “have,” “include,” or any similar expressions are intended to encompass non-exclusive inclusion. For example, an element or structure that comprises multiple components is not limited to only those components explicitly listed herein but may also include other components that are not expressly stated yet and inherently associated with the element or structure.

The remote electrical stimulation system of the present disclosure utilizes the cloud platform as a medium, enabling a physician to remotely provide a patient with the required electrical stimulation prescription, while allowing the patient to correspondingly perform remote electrical stimulation prescription therapy.

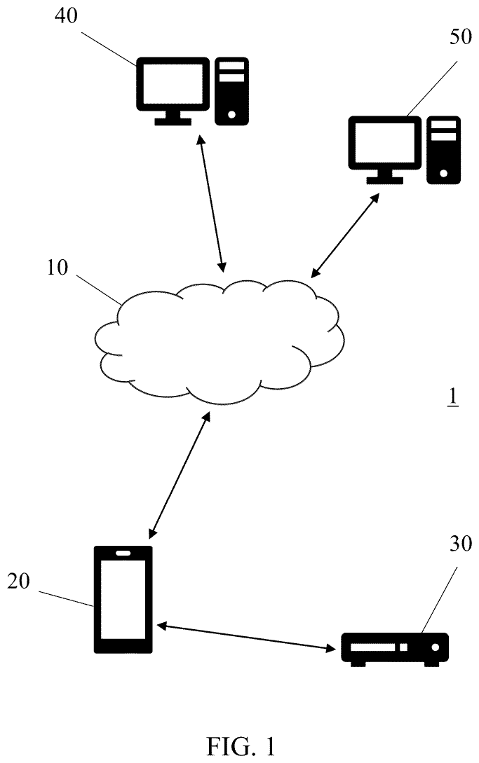

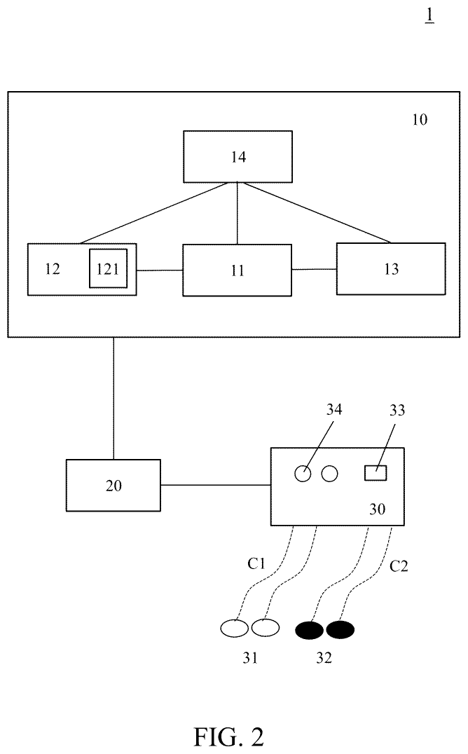

Please refer to FIG. 1 and FIG. 2 together, wherein FIG. 1 is a schematic diagram of the remote electrical stimulation system of the present disclosure, and FIG. 2 is a system block diagram of the remote electrical stimulation system of the present disclosure. As shown in FIG. 1 and FIG. 2, in at least one embodiment of the present disclosure, the remote electrical stimulation system 1 includes a cloud platform 10, a patient operating terminal 20, and an electrical stimulation device 30. In some embodiments of the present disclosure, the cloud platform 10 and the patient operating terminal 20 are connected to each other via a wireless network, and the patient operating terminal 20 and the electrical stimulation device 30 are also connected to each other via a wireless network.

In at least one embodiment of the present disclosure, the cloud platform 10 is configured to allow different users to log in and perform corresponding functions. In some embodiments of the present disclosure, the cloud platform 10 may be implemented as hardware (e.g., a computer host, a server, or a similar device), software (e.g., computer software or an application), or a combination of the aforementioned hardware and software. In some embodiments of the present disclosure, the cloud platform 10 includes a prescription database 11, a prescription editing module 12, and a prescription specifying module 13, wherein the aforementioned modules are electrically connected to each other.

In at least one embodiment of the present disclosure, the prescription database 11 is configured to store a plurality of electrical stimulation prescriptions for subsequent prescription editing, setting, comparison, learning, or other applications. In some embodiments of the present disclosure, the prescription database 11 may be a hardware device with a data storage function, such as a hard disk. However, the present disclosure is not limited thereto. For example, the prescription database 11 may also be a combination of a hardware device and associated data storage software. In some embodiments of the present disclosure, the plurality of electrical stimulation prescriptions may be preinstalled system-default electrical stimulation prescriptions or newly created electrical stimulation prescriptions stored by a user with the authorization for prescription editing.

In at least one embodiment of the present disclosure, an electrical stimulation prescription is defined as providing multiple carrier signals for the electrical stimulation device to execute the corresponding electrical stimulation treatment, wherein the electrical stimulation device may apply electrical stimulation to a corresponding body part of a patient based on the electrical stimulation prescription to achieve therapeutic effects. In some embodiments of the present disclosure, each electrical stimulation prescription includes a first carrier signal and a second carrier signal, wherein the first carrier signal and the second carrier signal may respectively provide continuous or discontinuous carrier waveforms or combinations thereof with different durations, waveforms, intensities, and frequencies. In at least one embodiment of the present disclosure, the first carrier signal and the second carrier signal belong to medium-to-low frequency carrier signals for treatment, rehabilitation, or symptom relief in the patient. However, the present disclosure is not limited thereto.

In at least one embodiment of the present disclosure, the prescription editing module 12 is configured to receive editing instructions input by a user with the authorization for prescription editing to edit or create an electrical stimulation prescription and store the processed electrical stimulation prescription in the prescription database 11. In some embodiments of the present disclosure, the prescription editing module 12 may be a combination of a hardware device with prescription editing functionality, such as a processor, and associated prescription editing software, wherein the aforementioned prescription editing software may be stored in a hardware device with a data storage function. However, the present disclosure is not limited thereto. For example, the prescription editing module 12 may also be a standalone software or hardware component.

In at least one embodiment of the present disclosure, since each patient's medical condition or physical status differs, an electrical stimulation prescription applied to the same medical condition may not always be fully applicable to different patients. Therefore, a user with the authorization for prescription editing, such as a professor doctor, has to determine an electrical stimulation prescription based on a patient's medical condition, physical status, or the symptoms of a particular disease. After logging into the cloud platform 10 via an editing operating terminal 40 (e.g., an operating device such as a computer, tablet, or mobile phone) through a wireless network, the user enters editing instructions into the generated prescription editing interface to select, edit, or adjust an electrical stimulation prescription. The processed electrical stimulation prescription is then uploaded to the cloud platform 10 and stored in the prescription database 11. In some embodiments of the present disclosure, the editing operating terminal 40 may pre-download and install an application from the cloud platform 10. After executing the application, the user may log into the cloud platform 10 via the user interface and access the relevant prescription editing interface.

In at least one embodiment of the present disclosure, the prescription specifying module 13 is configured to receive specifying instructions input by a user with the authorization for prescription specifying to designate one of a plurality of electrical stimulation prescriptions as an exclusive electrical stimulation prescription that can only be downloaded by the target patient, and to store the associated specifying data in the prescription database 11. In some embodiments of the present disclosure, the prescription specifying module 13 may be a combination of a hardware device with prescription specifying functionality, such as a processor, and associated prescription specifying software, wherein the aforementioned prescription specifying software may be stored in a hardware device with a data storage function. However, the present disclosure is not limited thereto. For example, the prescription specifying module 13 may also be a standalone software or hardware component.

As mentioned above, each patient's medical condition or physical status differs. Therefore, after logging into the cloud platform 10 via a specifying operating terminal 50 (e.g., an operating device such as a computer, tablet, or mobile phone) through a wireless network, the user with the authorization for prescription specifying (e.g., a clinic doctor) enters specifying instructions into the generated prescription specifying interface to select one of a plurality of electrical stimulation prescriptions and designate the selected electrical stimulation prescription as an exclusive electrical stimulation prescription that can only be downloaded by the target patient. In some embodiments of the present disclosure, the specifying operating terminal 50 may pre-download and install an application from the cloud platform 10. After executing the application, the user may log into the cloud platform 10 via the user interface and access the relevant prescription specifying interface.

In at least one embodiment of the present disclosure, the cloud platform 10 further comprises a login module 14. In some embodiments of the present disclosure, the login module 14 is configured to allow a user to log in and identify the logged-in user's identity, so as to correspondingly grant the authorization for prescription editing, the authorization for prescription specifying, and/or the authorization for downloading the exclusive electrical stimulation prescription to the user based on the user's identity. In other words, different users (e.g., physicians or patients) may have different permissions and functionalities after logging into the cloud platform 10. In some embodiments of the present disclosure, the login module 14 may be software executed by a processor with user login and data processing functionality, wherein the aforementioned software may be stored in a hardware device with a data storage function. However, the present disclosure is not limited thereto. For example, the login module 14 may also be a combination of hardware and software.

In some embodiments of the present disclosure, when the login module 14 determines that the logged-in user has the authorization for prescription editing, the login module 14 notifies the prescription editing module 12 to generate a prescription editing interface and display it on the editing operating terminal 40. Accordingly, the user with the authorization for prescription editing may enter editing instructions via the prescription editing interface, and once the prescription editing interface receives the editing instructions, it may edit or create an electrical stimulation prescription based on the editing instructions. After the editing or creation of the electrical stimulation prescription is completed, it is uploaded and stored in the cloud platform 10.

In some embodiments of the present disclosure, when the login module 14 determines that the logged-in user has the authorization for prescription specifying, the login module 14 notifies the prescription specifying module 13 to generate a prescription specifying interface and display it on the specifying operating terminal 50. Accordingly, the user with the authorization for prescription specifying may enter specifying instructions via the prescription specifying interface, and once the prescription specifying interface receives the specifying instructions, it may designate an exclusive electrical stimulation prescription that can only be downloaded by the target patient based on the specifying instructions. The associated specifying data may then be stored in the prescription database 11.

In at least one embodiment of the present disclosure, the patient operating terminal 20 allows personnel with the authorization for downloading the exclusive electrical stimulation prescription to download the exclusive electrical stimulation prescription from the cloud platform 10. In some embodiments of the present disclosure, subject to current medical regulations, personnel with the authorization for downloading the exclusive electrical stimulation prescription includes physicians and other medical-related personnel. In other embodiments of the present disclosure, once telemedicine is approved for relevant use in the future, personnel with the authorization for downloading the exclusive electrical stimulation prescription may further include the target patients themselves. In some embodiments of the present disclosure, the patient operating terminal 20 may be an electronic operating device, such as a computer, tablet, or mobile phone, wherein the patient operating terminal 20 may pre-download and install an application from the cloud platform 10. After executing the application, the user may log into the cloud platform 10 via an operating interface and access the relevant interface, thereby downloading the designated exclusive electrical stimulation prescription applicable to the target patient from the cloud platform 10. In some embodiments of the present disclosure, the downloaded exclusive electrical stimulation prescription may be stored in the patient operating terminal 20. Additionally, the target patient may also fill out and submit a questionnaire via the aforementioned operating interface to the cloud platform 10, thereby providing feedback for physician reference or for subsequent adjustments to the electrical stimulation prescription.

In at least one embodiment of the present disclosure, the electrical stimulation device 30 may receive the exclusive electrical stimulation prescription from the patient operating terminal 20 and utilize the exclusive electrical stimulation prescription to perform an electrical stimulation treatment on the target patient. In some embodiments of the present disclosure, the electrical stimulation device 30 may be a commonly used electrical therapy device with a patch set or a device with a similar electrical stimulation function. As shown in FIG. 2, in at least one embodiment of the present disclosure, the electrical stimulation device 30 includes a first patch set 31 corresponding to a first channel C1 and a second patch set 32 corresponding to a second channel C2. When the electrical stimulation device 30 receives the exclusive electrical stimulation prescription and begins executing the electrical stimulation treatment, the electrical stimulation device 30 transmits the first carrier signal of the exclusive electrical stimulation prescription to the first patch set 31 via the first channel C1 and transmits the second carrier signal of the exclusive electrical stimulation prescription to the second patch set 32 via the second channel C2. In some embodiments of the present disclosure, the first patch set 31 and the second patch set 32 each include two patches, and the first patch set 31 and the second patch set 32 may be attached to different body parts of the target patient, thereby allowing the first carrier signal and the second carrier signal of the exclusive electrical stimulation prescription to be applied for electrical stimulation therapy or rehabilitation targeting the target patient.

In at least one embodiment of the present disclosure, the first carrier signal and the second carrier signal of the exclusive electrical stimulation prescription may correspond to different carrier waveform combinations and/or timing sequences. In some embodiments of the present disclosure, since the first carrier signal and the second carrier signal provide different carrier waveform combinations and timing sequences, the first patch set 31 and the second patch set 32 may generate electrical stimulation effects in different circuits based on the first carrier signal and the second carrier signal, respectively. For example, the timing sequences of the carrier waveforms corresponding to the first channel C1 and the second channel C2 may be staggered, or base waveforms may be cross-combined, allowing the first carrier signal and the second carrier signal to provide different carrier waveforms at the same time. Therefore, when the target patient attaches the first patch set 31 and the second patch set 32 to different muscle regions, electrical stimulation can be applied to different muscles accordingly.

Taking arm muscles as an example, in at least one embodiment of the present disclosure, the first patch set 31 and the second patch set 32 may be attached to the anterior and posterior muscles of the arm, respectively. By applying different electrical stimulation effects, movements such as arm extension and contraction can be achieved, thereby facilitating rehabilitation therapy for stroke patients or patients with muscle disorders.

In some embodiments of the present disclosure, the electrical stimulation device 30 may be incorporated with a built-in control chip, memory, amplifier, and network transmission module, among other electronic components, to facilitate the reception of the exclusive electrical stimulation prescription and execute corresponding electrical stimulation accordingly. As shown in FIG. 2, in some embodiments of the present disclosure, the electrical stimulation device 30 may further include a power toggle button 33 and an intensity adjustment button 34, wherein the power toggle button 33 is configured to switch the power supply of the electrical stimulation device 30 on or off. Additionally, the intensity adjustment button 34 may be configured as two separate buttons to respectively adjust the carrier signal intensity of the first carrier signal output by the first patch set 31 and the second carrier signal output by the second patch set 32. Furthermore, in some embodiments of the present disclosure, the patient may also communicate with the electrical stimulation device 30 via the operating interface of the patient operating terminal 20 to control the electrical stimulation device 30 to start or stop executing the electrical stimulation treatment, or control the carrier signal intensity of the first carrier signal output by the first patch set 31 and the second carrier signal output by the second patch set 32.

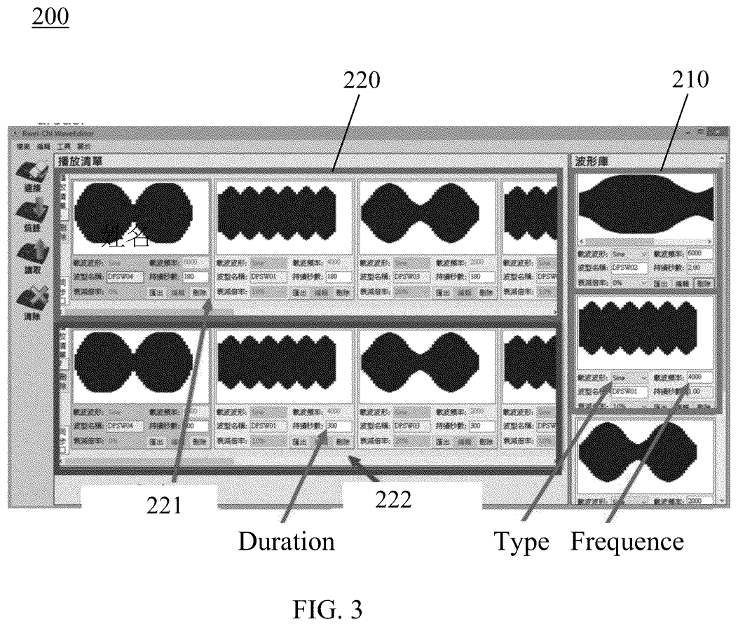

Please refer to FIGS. 1 to 3 together, wherein FIG. 3 is a schematic diagram of the prescription editing interface generated by the remote electrical stimulation system of the present disclosure. As shown in FIGS. 1 to 3, in at least one embodiment, the remote electrical stimulation system 1 of the present disclosure may generate a prescription editing interface 200 via the prescription editing module 12. In some embodiments of the present disclosure, a user with the authorization for prescription editing may execute an application on the editing operating terminal 40, enter a registered account and password, and log into the cloud platform 10. When the login module 14 determines that the logged-in user has the authorization for prescription editing, the login module 14 notifies the prescription editing module 12 to generate the prescription editing interface 200 and display it on the editing operating terminal 40. In some embodiments of the present disclosure, the user may then operate the prescription editing interface 200 to edit or create an electrical stimulation prescription. The aforementioned process involves conventional account and interface processing techniques, which are not subject to any special restrictions in the present disclosure and thus will not be described in further detail.

As shown in FIG. 2, in some embodiments of the present disclosure, the prescription editing module 12 includes a carrier waveform library 121, which is configured to store a plurality of carrier waveform templates that may be used as carrier signals in an electrical stimulation prescription. In some embodiments of the present disclosure, the prescription editing interface 200 includes a waveform selection area 210 and an editing area 220, wherein the waveform selection area 210 is configured to display a plurality of carrier waveform templates stored in the carrier waveform library 121 and to receive editing instructions for adjusting or modifying the settings of the carrier waveform templates. In some embodiments of the present disclosure, the waveform selection area 210 may allow arbitrary adjustment or modification of carrier waveform parameters for each displayed carrier waveform template, such as type (e.g., sine wave, square wave, or triangular wave), frequency (e.g., between 2 kHz and 8 kHz), or attenuation ratio. However, the present disclosure is not limited thereto.

In some embodiments of the present disclosure, the editing area 220 is configured to edit any electrical stimulation prescription. For example, the editing area 220 may receive editing instructions to select a single or multiple carrier waveform templates from the waveform selection area 210 based on the editing instructions and export them to the editing area 220 for editing. This allows the design or adjustment of different carrier signals in an electrical stimulation prescription using a single carrier waveform template or a combination of multiple carrier waveform templates. In some embodiments of the present disclosure, the editing area 220 includes a first carrier signal area 221 and a second carrier signal area 222, wherein the first carrier signal area 221 is configured to display horizontally the first carrier signal of a single electrical stimulation prescription along a time axis, allowing each carrier waveform in the first carrier signal to be edited with respect to its corresponding position and duration (e.g., in seconds) on the time axis. Similarly, the second carrier signal area 222 is configured to display horizontally the second carrier signal of a single electrical stimulation prescription along the time axis, allowing each carrier waveform in the second carrier signal to be edited with respect to its corresponding position and duration (e.g., in seconds) on the time axis.

Additionally, in some embodiments of the present disclosure, if the waveforms stored in the built-in waveform library of the software do not meet the requirements of a user with the authorization for prescription editing, the editing area 220 of the prescription editing interface may be switched to a drawing interface with an equidistant grid, allowing the user to input editing instructions and manually draw an ideal new carrier waveform using the drawing interface, which can serve as a different carrier signal in a single electrical stimulation prescription. In some embodiments of the present disclosure, the user may also create a new carrier waveform by adjusting different waveform parameters. In some embodiments of the present disclosure, the user may further utilize the equidistant grid provided by the drawing interface to intuitively draw an ideal new carrier waveform in a graphical manner by filling in the spaces formed by the equidistant grid, thereby eliminating the tedious steps of repeatedly adjusting parameters to generate a new carrier waveform. Once the drawing is complete, the new carrier waveform may be displayed in the editing area 220 and/or stored in the waveform selection area 210 for subsequent use by the user.

When performing an electrical stimulation treatment on a patient's muscle region, since human muscles have memory, it may result in standardized electrical stimulation if an electrical stimulation prescription can only provide a carrier signal with a single waveform or frequency, causing the muscles to gradually adapt to similar stimuli that reduces the effectiveness of the electrical stimulation. Accordingly, if an electrical stimulation prescription can provide variations in carrier waveform combinations, it can effectively deliver different types of stimulation to the muscles, thereby maintaining the effectiveness of rehabilitation therapy.

In some embodiments, the remote electrical stimulation system 1 of the present disclosure may allow a user with the authorization for prescription editing to edit or create an electrical stimulation prescription via the prescription editing interface 200 generated by the prescription editing module 12 of the cloud platform 10. In some embodiments of the present disclosure, the user with the authorization for prescription editing may freely select the required carrier waveform from a variety of carrier waveform templates provided by the prescription editing interface 200, and export it to the editing area 220 for carrier waveform editing or combination, thereby forming different carrier signals in an electrical stimulation prescription. In other words, the user with the authorization for prescription editing may design an electrical stimulation prescription suitable for a patient's condition based on their needs or personal experience.

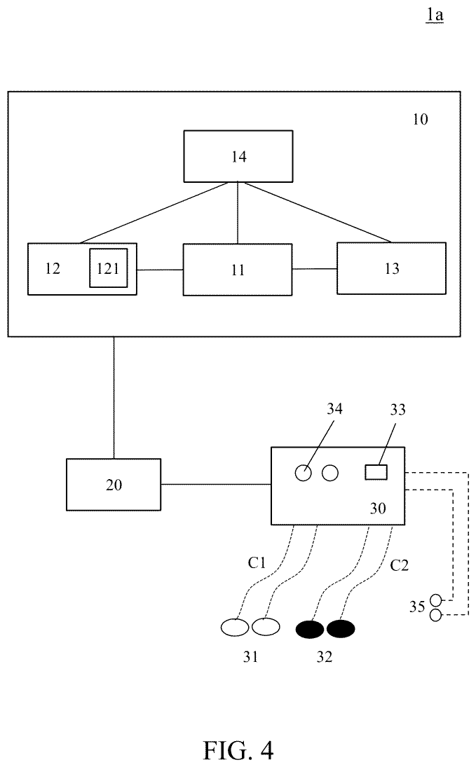

Please also refer to FIG. 4, which is a system block diagram of another embodiment of the remote electrical stimulation system of the present disclosure. As shown in FIG. 4, in at least one embodiment of the present disclosure, the electrical stimulation device 30 of the remote electrical stimulation system 1a may further include a plurality of sensors 35. In some embodiments of the present disclosure, the plurality of sensors 35 are configured to obtain feedback information when the target patient executes an electrical stimulation treatment using the exclusive electrical stimulation prescription. Such feedback information may include physiological parameters, such as heart rate, body temperature, blood pressure, or muscle vibration, and is transmitted back to the cloud platform 10 via the patient operating terminal 20. In some embodiments of the present disclosure, before executing an electrical stimulation treatment, the target patient may first attach the first patch set 31 and the second patch set 32 of the electrical stimulation device 30 to muscle regions to be stimulated. Based on the physiological parameters to be measured, the plurality of sensors 35 may be placed at appropriate locations (e.g., near the muscle regions or at positions suitable for sensing physiological parameters), enabling the collection of relevant feedback information during the execution of the electrical stimulation treatment using the exclusive electrical stimulation prescription. In some embodiments of the present disclosure, once the feedback information is collected and transmitted back to the cloud platform 10, it may serve as a reference for a user with the authorization for prescription specifying to readjust or replace the exclusive electrical stimulation prescription.

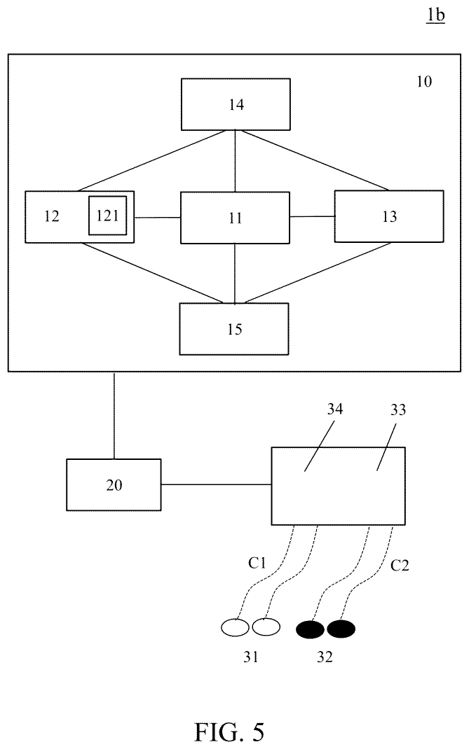

Please also refer to FIG. 5, which is a system block diagram of yet another embodiment of the remote electrical stimulation system of the present disclosure. As shown in FIG. 5, in at least one embodiment of the present disclosure, the cloud platform 10 of the remote electrical stimulation system 1b may further include a learning module 15. In some embodiments of the present disclosure, the learning module 15 may compile historical records of each specified exclusive electrical stimulation prescription along with the corresponding medical history data of the target patient and perform comparative learning. This allows the prescription specifying interface to provide recommendation information for selecting an exclusive electrical stimulation prescription for different patients.

In some embodiments of the present disclosure, the learning module 15 may be a combination of a hardware device with data learning capabilities, such as a processor, and related data learning software, such as an artificial intelligence model or an artificial neural network architecture. However, the present disclosure is not limited thereto, and the learning module 15 may also be implemented as a standalone software or hardware module. In some embodiments of the present disclosure, the learning module 15 performs comparative learning based on the exclusive electrical stimulation prescriptions and the medical conditions of the target patient. During the process in which a user with the authorization for prescription specifying enters specifying instructions via the prescription specifying interface to designate an exclusive electrical stimulation prescription, the learning module 15 provides recommendation information for selecting an exclusive electrical stimulation prescription at the prescription specifying interface, thereby assisting the user with the authorization for prescription specifying in more quickly and conveniently responding to different patient conditions to select or adjust a more suitable electrical stimulation prescription, which is then designated as the exclusive electrical stimulation prescription.

Accordingly, the remote electrical stimulation system of the present disclosure utilizes the cloud platform as a medium, allowing the patient to obtain the exclusive electrical stimulation prescription specified by a physician to perform remote electrical stimulation therapy, thereby reducing the number of visits and time required for treatment at a medical institution. Users with different authorizations (e.g., physicians or patients) can interact with the cloud platform via an electronic device, such as a mobile phone or a computer, through a network to individually perform functions such as prescription editing, prescription specifying, or downloading of the exclusive electrical stimulation prescriptions.

The above-described embodiments are fundamentally for illustrative purposes only and are not intended to limit the embodiments of the claimed subject matter or their applications. Furthermore, although at least one exemplary embodiment has been provided in the foregoing description, it should be understood that numerous variations of the present disclosure may exist. Likewise, it should be understood that the embodiments described herein are not intended to limit the scope, applications, or configurations of the claimed subject matter in any way. On the contrary, the above-described embodiments are intended to provide a straightforward guide for those skilled in the art to implement one or more embodiments described herein. Moreover, various modifications may be made to the functions and arrangements of components without departing from the scope defined by the claims, and the scope of the claims encompasses known equivalents as well as all foreseeable equivalents at the time of filing of this patent application.

What is claimed is:

1. A remote electrical stimulation system, comprising:
a cloud platform including a prescription database, a prescription editing module, and a prescription specifying module, wherein the prescription database is configured to store a plurality of electrical stimulation prescriptions, the prescription editing module is configured to receive editing instructions to edit or create an electrical stimulation prescription, and the prescription specifying module is configured to receive specifying instructions to designate one of the plurality of electrical stimulation prescriptions as an exclusive electrical stimulation prescription downloadable by a target patient having download authorization for the exclusive electrical stimulation prescription;
a patient operating terminal connected to the cloud platform and configured to allow authorized personnel to assist the target patient in downloading the exclusive electrical stimulation prescription from the cloud platform; and
an electrical stimulation device connected to the patient operating terminal and configured to receive the exclusive electrical stimulation prescription and execute an electrical stimulation treatment on the target patient based on the exclusive electrical stimulation prescription.

2. The remote electrical stimulation system according to claim 1, wherein the electrical stimulation device includes a first patch set corresponding to a first channel and a second patch set corresponding to a second channel, and the exclusive electrical stimulation prescription includes a first carrier signal and a second carrier signal.

3. The remote electrical stimulation system according to claim 2, wherein the electrical stimulation device is configured to transmit the first carrier signal to the first patch set via the first channel and transmit the second carrier signal to the second patch set via the second channel.

4. The remote electrical stimulation system according to claim 3, wherein the first carrier signal and the second carrier signal correspond to different carrier waveform combinations and/or timing sequences.

5. The remote electrical stimulation system according to claim 3, wherein the first carrier signal and the second carrier signal correspond to different carrier waveforms at the same time.

6. The remote electrical stimulation system according to claim 1, wherein the cloud platform further comprises a login module configured to allow a user to log in and identify identity of the logged-in user to correspondingly grant at least one of an authorization for prescription editing, an authorization for prescription specifying, and an authorization for downloading the exclusive electrical stimulation prescription.

7. The remote electrical stimulation system according to claim 6, wherein the login module is configured to determine that the user has the authorization for prescription editing.

8. The remote electrical stimulation system according to claim 7, wherein the prescription editing module is configured to generate a prescription editing interface and receive the editing instructions via the prescription editing interface to edit or create the electrical stimulation prescription.

9. The remote electrical stimulation system according to claim 8, wherein the prescription editing module includes a carrier waveform library, and the prescription editing interface includes a waveform selection area and an editing area.

10. The remote electrical stimulation system according to claim 9, wherein the waveform selection area is configured to display carrier waveform templates stored in the carrier waveform library and to adjust or modify settings of any of the carrier waveform templates based on the editing instructions, and wherein the editing area is configured to select and edit either a single carrier waveform template or a combination of multiple carrier waveform templates from the waveform selection area based on the editing instructions or configured to draw a new carrier waveform based on the editing instructions.

11. The remote electrical stimulation system according to claim 10, wherein the waveform selection area is configured to set at least one of type, frequency, and attenuation ratio of the carrier waveform for each displayed carrier waveform template.

12. The remote electrical stimulation system according to claim 10, wherein the editing area includes a first carrier signal area and a second carrier signal area.

13. The remote electrical stimulation system according to claim 12, wherein the first carrier signal area is configured to display, along a horizontal time axis, the first carrier signal of the electrical stimulation prescription and allow editing of each carrier waveform in the first carrier signal for a corresponding position and duration on the time axis.

14. The remote electrical stimulation system according to claim 13, wherein the second carrier signal area is configured to display, along the time axis, the second carrier signal of the electrical stimulation prescription and allow editing of each carrier waveform in the second carrier signal for a corresponding position and duration on the time axis.

15. The remote electrical stimulation system according to claim 6, wherein the login module is configured to determine that the user has the authorization for prescription specifying.

16. The remote electrical stimulation system according to claim 15, wherein the prescription specifying module is configured to generate a prescription specifying interface and receive the specifying instructions via the prescription specifying interface to specify the exclusive electrical stimulation prescription downloadable by the target patient.

17. The remote electrical stimulation system according to claim 16, wherein the cloud platform further comprises a learning module configured to compile historical records of each specified exclusive electrical stimulation prescription and corresponding medical history data of the target patient for comparative learning and provide recommendation information for selecting the exclusive electrical stimulation prescription in the prescription specifying interface for different patients.

18. The remote electrical stimulation system according to claim 1, wherein the electrical stimulation device further comprises a plurality of sensors configured to obtain feedback information from the target patient in using the exclusive electrical stimulation prescription to perform the electrical stimulation treatment and configured to transmit the feedback information back to the cloud platform via the patient operating terminal.
